(19) United States
(12) Patent Application Publication
(10) Pub. No.: US 2010/0233221 A1
(43) Pub. Date: Sep. 16, 2010

(54) STABLE DOUBLE EMULSIONS

(75) Inventors: Britta Folmer, Lausanne (CH); Martin Michel, Lausanne (CH); Cecile Gehin-Delval, Labergement Sainte Marie (FR); Simone Acquistapace, La Tour-de-Peilz (CH); Martin Leser, Bretigny-s/Morrens (CH); Axel Syrbe, Clarens (CH); Sebastien Marze, Lausanne (CH)

Correspondence Address: K&L Gates LLP, P.O. Box 1135, CHICAGO, IL, 60690, US

(73) Assignee: NESTEC S.A., Vevey (CH)

(21) Appl. No.: 12/666,069

(22) PCT Filed: Jun. 27, 2008
(86) PCT No.: PCT/EP08/58318

§ 371 Date: May 17, 2010

(30) Foreign Application Priority Data
Jun. 29, 2007 (EP) 07111403.7

(51) Int. Cl. A23D 7/00; A61K 8/06; A61K 9/113; A23D 7/005

(52) U.S. Cl. 424/401; 424/400; 426/602; 426/589

(57) ABSTRACT

The invention relates to double emulsions, in particular double emulsions of the water-in-oil-in-water type, which are organoleptically similar to full-fat oil in water emulsions and which are stabilised by mixture of emulsifiers. The invention also relates to a method for producing said double emulsions, and to the use of a mixture of emulsifier for stabilising said emulsions.

### FIELD OF THE INVENTION

The invention relates to double emulsions, in particular double emulsions of the water-in-oil-in-water type, which are organoleptically similar to full-fat oil in water emulsions and which are stabilised by a selection of emulsifiers. The invention also relates to a method for producing said double emulsions, and to the use of a selection of emulsifier for stabilising said emulsions.

### BACKGROUND OF THE INVENTION

Double emulsions, also named multiple emulsions, can be considered as an emulsion of an emulsion: the oil droplets which are dispersed in an aqueous phase, contain themselves small water droplets. Water-in-oil-in-water (W/O/W) emulsions are interesting for fat reduction to create low fat products. Indeed, the oil droplets in an ordinary oil-in-water emulsion can be replaced by droplets made of a water-in-oil emulsion. Low-fat products obtained with multiple emulsions are described in EP 0 711 115, US 2004/0101613 (EP 1 565 076), EP 0 345 075. Multiple emulsions are also often used in cosmetic applications. For instance, EP 1 097 702, EP 0 614 660, EP 0 650 352, EP 0 648 102, EP 0 507 693 all describe cosmetic compositions in the form of water in oil in water emulsions.

The main problems encountered with double emulsions is their stability. Most solutions proposed to date rely on the proper choice of the emulsifiers used. For instance, EP 0 731 685 describes a stable multiple emulsion obtained by using emulsifiers having a HLB value less than 6. EP 0 631 774 also describes storage stable multiple emulsions comprising particular hydrophobic and hydrophilic emulsifiers. WO 03/049553 similarly relates to stable multiple emulsions obtained by selecting the appropriate emulsifiers used for the internal water-in-oil emulsion and for the external oil-in-water emulsion. There is however still room for improvement. The FR Patent 2823450 concerns a final product which is a suspension, that means a solid product and not a liquid Water-in-oil-in-water emulsion. This is not a food product, but only a product for use in cosmetic. The WO patent application 03/049553 concerns a double emulsion in which the inner water phase contains a viscosifier, such as alginate. In this case, the objective is to obtain a gelified product, which is not the case for us.

### OBJECT OF THE INVENTION

The object of the present invention is thus to provide stabilised double emulsions which can be used in a number of applications.

### SUMMARY

Accordingly, the present object is achieved by means of the features of the independent claims. The dependent claims develop further the central idea of the invention.

In a first aspect, the invention relates to a double emulsion comprising an internal aqueous phase dispersed in an oil phase forming a water-in-oil emulsion, said water-in-oil emulsion being dispersed in an external aqueous phase, wherein the water-in-oil emulsion comprises at least one emulsifier, wherein at least the internal aqueous phase comprises solutes and wherein the external aqueous phase comprises at least one hydrophilic polymer or polymer aggregates.

A method for the preparation of a double emulsion comprising the steps of:
a. Preparing an internal aqueous phase comprising solutes
b. Combining the internal aqueous phase with an oil phase comprising at least one emulsifier to obtain a stabilised water-in-oil emulsion
c. Combining the water-in-oil emulsion with an external aqueous phase to form a double emulsion,
   wherein the external aqueous phase comprises at least one hydrophilic polymer or polymer aggregates, and the double emulsion obtainable by said method both form part of the present invention.

A further aspect of the invention relates to the use of at least one emulsifier for stabilising a water-in-oil emulsion, wherein the water-in-oil emulsion is part of a double emulsion comprising said water-in-oil emulsion dispersed in an external aqueous phase.

Finally, the use of the double emulsion of the invention in food products, clinical products, pharmaceutical products, nutricosmetics, cosmetics, agro-chemical or other industrial products is also part of the invention.

### FIGURES

The present invention is further described hereinafter with reference to some of its embodiments shown in the accompanying drawings in which:

FIG. 1 represents light microscopy pictures (DIC) of the double emulsion at different time points.

FIGS. 2a and 2b shows the rheological behaviour of the double emulsion of the invention

FIG. 3 shows the influence of salt in the internal aqueous phase on the stability of the double emulsion

### DETAILED DESCRIPTION OF THE INVENTION

The double emulsions of the invention are preferably emulsions of the water-in-oil-in-water type. They comprise an aqueous phase (the inner aqueous phase) dispersed in an oil phase forming a water-in-oil emulsion. Said water-in-oil emulsion is dispersed in an external aqueous phase.

According to the invention, the inner aqueous phase is dispersed and stabilised by means of an emulsifier or emulsifier mixture in an oil phase. The inner aqueous phase also comprises solutes.

In one embodiment, the water-in-oil emulsion comprises at least two different emulsifiers having different molecular weights.

By emulsifiers having differing molecular weights is preferably to be distinguished between emulsifiers having a molecular weight of less than 2000 g/mol and emulsifiers having a molecular weight of more than 700 g/mol. In a preferred embodiment, at least one emulsifier has a molecular weight of less than 1800 g/mol, more preferably less than 1500 g/mol, even more preferably less than 1200 g/mol, while at least a second emulsifier has a molecular weight of more than 800 g/mol, preferably more than 1000 g/mol, even more preferably more than 1200 g/mol. Such emulsifiers may be termed “low-molecular weight” emulsifiers and “high-molecular weight” emulsifiers respectively. Preferably, the “low-molecular weight” emulsifier has a molecular weight lower than the “high-molecular weight” emulsifier.

According to a preferred embodiment, at least one of the emulsifier is selected from the group of low molecular weight emulsifiers consisting of fatty acids, sorbitan esters, propylene glycol mono- or diesters, pegylated fatty acids, monoglycerides, derivatives of monoglycerides, diglycerides, pegylated vegetable oils, polyoxyethylene sorbitan esters, phospholipids, lecithin, cephalin, lipids, galactolipids, sugar esters, sugar ethers, sucrose esters, sorbitol anhydride monostearate, sorbitol anhydride monooleate, glycerol monooleate, or mixtures thereof and at least a second emulsifier is selected from the group of high molecular weight emulsifiers consisting of polyglycerol esters, polyglycerol polyricinoleic acid (PGPR), cellulose and its derivatives such as ethylcellulose, oil soluble proteins or peptides or hydrolysates, protein-polysaccharide complexes, coacervates or conjugates, food particles, fat particles, solid-lipid nanoparticles, micronised nutrient crystals, dietary fibres or mixtures thereof.

Accordingly, the emulsifier mixture preferably comprises at least one “low-molecular weight” emulsifier and at least one “high-molecular weight” emulsifier.

If only one emulsifier is used, it is possible to use either the “low molecular weight” emulsifier or the “high molecular weight” emulsifier, preferably the “high molecular weight” emulsifier.

Under protein-polysaccharide complexes, coacervates or conjugates, which can be used as a “high molecular weight” emulsifier in the invention is meant any protein-polysaccharide mixtures which forms interfacially active supra-molecular aggregates, which are either physical complexes or which are chemically linked together via chemical bonds. These coacervates or conjugates have the properties of accumulating at the water-oil interface, reducing the interfacial tension and helping to disperse the water droplets into an oil phase and to stabilise the obtained water-in-oil emulsion (w/o emulsion).

Furthermore, the food particles which can be used in the present invention as part of the “high-molecular weight” emulsifier include seeds, spices, seasonings, spores, cloves, pepper, fennel, cumin, coriander, nutmeg, poppy grains, paprika, cinnamon, talcum, pollen of flowers, wheat germs, wheat bran, saffron, coconut, cacao, melanoidins, sugar crystals, protein aggregates, ginger, curry, titanium dioxide polymeric particles, calcium carbonate polymeric particles, microcrystalline cellulose, or a mixture thereof. The particles may already be surface active on their own or may get their surface active properties after grinding of the particle material and/or by addition of a “low molecular weight” emulsifier which adsorbs onto the surface of the particles. The interfacial activity of the particles of this invention (adsorption onto the water-oil interface or desorption from the water-oil interface) can be followed using classical surface tensiometry, such as the Wilhelmy plate of Drop shape or Drop volume or Bubble pressure tensiometry (R. Miller et al., SÖFW-Journal 130, 2-10 (2004)). The adsorption/attachment of the interfacially active particles to the water droplets can be followed by light and/or electron microscopy. Especially polarized light or fluorescent microscopy is a suitable technique to visualise the attachment of interfacially active particles at the air-bubble surface.

The term ‘interfacially active particles’ is used herein to describe colloidal particles, i.e. supra-molecular aggregates, having a diameter between 0.5 nm up to 100 microns, preferably 0.5-50 microns, which act in many ways like emulsifiers in the sense that they are able to adsorb or attach to a water-oil interface. The unique feature of the adsorbed particles is that their attachment at the water-oil interface is irreversible. This is clearly not observed using commonly used emulsification agents, such as low molecular surfactants, which are adsorbed in a reversible way and desorb again after a certain time (an adsorption/desorption equilibrium between the water-oil is established). The irreversible attachment of the particles to the water-oil interface gives the water droplets the remarkable stability against coalescence or Ostwald ripening.

According to the present invention, the interfacially active particles can be created in different ways: one way to create interfacially active particles is by using “low molecular weight” emulsifiers, heating the aqueous dispersion above their Krafft temperature and cooling the dispersion down again to room temperature while stirring. During the cooling step, the particles are formed. The emulsification of the internal aqueous phase into the oil phase can be done during the cooling step or just after the system is cooled down to room temperature. The used emulsifier can be a mixture of different emulsifiers or can be used singly.

Similarly fat particles can be made from fat sources selected from sunflower oil, palm oil, rapeseed oil, cotton seed oil, soy bean oil, maize oil, Shea oil, cocoa butter, or fractions thereof or in their hardened form or as fraction of the hardened oil or as partially hydrolysed oil rich in diglycerides or monoglycerides or as mixtures thereof. The particles are micronised and their interfacial activity and emulsification properties can be tuned by adding a “low molecular weight” emulsifier, similarly as described above for creating interfacially active food particles.

Under solid-lipid nanoparticles is to be understood particulate systems with mean particle diameters ranging from 50 up to 1000 nm. They are mainly known as drug delivery systems and formed by homogenisation as described by Dong Zhi Hou, Chang Sheng Xie, Kai Jin Huang, Chang Hong Zhu in Biomaterials 24 (2003) 1781-1785. Their interfacial activity and emulsification properties can be tuned by adding a “low molecular weight” emulsifier, similarly as described above for creating interfacially active food particles.

Finally, micronised nutrient crystals such as phytosterols, hesperidin, or lycopene crystals can also be used as one of the “high-molecular weight” emulsifier. It concerns all nutrients which are insoluble both in water and oil phases and, as a consequence, forming crystals. Their interfacial activity and emulsification properties can be tuned by adding a “low molecular weight” emulsifier, similarly as done for creating interfacially active food particles.

Most preferably, the mixture of emulsifiers used to stabilise the inner water-in-oil emulsion is a mixture of PGPR and glycerol monooleate (GMO). It is also possible to use only PGPR or only GMO.

It has been found that using simply a “low-molecular weight” emulsifier, or alternatively using simply a “high-molecular weight” emulsifier can be enough to stabilise the inner water/oil interface. By using a mixture of emulsifiers according to the present invention, a product having the desired consistency and stability is obtained.

According to a preferred embodiment, the external aqueous phase comprises a hydrophilic polymer or polymer aggregates. Thus, the oil droplets (which contain the dispersed inner aqueous phase) are stabilised in the external aqueous phase. Examples of such polymers or aggregates of polymers are amidated low methoxy pectin, caseinate, whey proteins, milk proteins, egg proteins, egg yolk, soy proteins, acacia gum, starch derivatives or other o/w emulsion stabilizing proteins or polysaccharides, or hydrophilic particles made out of protein aggregates, polysaccharide aggregates, hydrophilic particles made out of protein-surfactant aggregates, hydrophilic particles made out of protein-polysaccharide mixtures, hydrophilic particles made out of polysaccharide-polysaccharide mixtures, hydrophilic particles made out of polysaccharide-protein phase separating mixtures, or any mixtures thereof.

By hydrophilic particles is meant particles which are dispersible into an aqueous phase and show an interfacial activity. The term “interfacially active particles” is as defined above.

Preferably, protein-polysaccharide mixtures, coacervates, conjugates, hybrids or particles are used as hydrophilic polymer in the present invention.

Most preferably, the hydrophilic polymer is taken in the group consisting of whey protein isolate, amidated low methoxy pectin, egg yolk or a mixture thereof.

The inner aqueous phase preferably comprises solutes selected from salts, polyols and/or sugars. Any water soluble salts, including organic salts, polyols or sugars can be used. Salts may be, for instance, sodium chloride, potassium chloride, calcium chloride, magnesium chloride, zinc chloride etc. Polyols or sugars which can be used in the present invention comprise any mono-, di- and oligosaccharides, starch, degradation products of starch, maltodextrins, dextran, glucose, sucrose, fructose, glycerol, polyglycerol, ethyleneglycol, propylene glycol etc.

The presence of solutes in the inner aqueous phase creates initially an osmotic pressure gradient between the inner aqueous phase and the outer aqueous phase. Thus, the internal water-in-oil emulsion of the present invention is under osmotic pressure. This causes a swelling of the inner aqueous phase over time such that a textured double emulsion may be obtained. This is only achieved when the inter water-oil interface is efficiently stabilised by the used emulsifiers. The texture and consistency of the double emulsion of the invention can be modulated and regulated from low viscous (pourable) to highly viscous (spreadable) by, for instance, choosing a suitable type and amount of solute.

Thus, by the present invention, it is not necessary to introduce a gelling agent i.e. a viscosifier at concentrations above the gel point, in the external aqueous phase to obtain the desired texture and consistency. The external aqueous phase is not gelified. If a gelling agent would be present, it is present in the amount of less than 5%, preferably of less than 2%.

Furthermore, referring to FIG. 3, it can be seen that the presence of solutes (e.g. salt) in the inner aqueous phase has an impact on the stability of the double emulsion. The light microscopy images show the difference in stability directly after preparation (1), 1 hour after preparation (2), 3-7 days after preparation (3) for a double emulsion comprising salt in the inner water phase (A) and a double emulsion not comprising salt in the inner water phase (B). The images show that A is very stable over time whereas B is not stable at all.

The double emulsion of the present invention may comprise from 1 to 55%, preferably 2 to 45%, more preferably 3 to 35% oil. This depends on the desired product. For instance, by varying the amount of oil, the double emulsions of the invention may vary in consistency and texture from fluid, to viscous, to full-fat mayonnaise-like textures.

Another way to vary the final product consistency is by varying the amount of aqueous solute phase droplets in the inner w/o emulsion. Increasing the amount of the aqueous solute phase in the w/o emulsion transforms the consistency and texture of the final double emulsion of the invention from fluid to viscous and mayonnaise-like while keeping the overall fat content in the double emulsion constant.

In one embodiment, a viscosifier is also present in the double emulsion in order to fine tune the final texture and consistency of the double emulsion product. Preferably, the viscosifier is present in the external aqueous phase. The presence of a viscosifier may enhance the viscosity of said product and the stability of the emulsion against creaming and/or phase separation. This is particularly advantageous when low-fat double emulsions are produced.

Viscosifiers which can be used in the present invention may be selected from amidated low methoxy pectin, pectin, alginates, carrageenan, locust bean gum, guar gum, tragacanth, Acacia gum, xanthan gum, Karaya gum, gellan gum, polydextrose, dextrin, modified starch, oxidized starch, cellulose derivatives, or mixtures thereof.

When amidated low methoxy pectin is already used as part of the hydrophilic polymer for stabilising the oil phase in the external aqueous phase, a further viscosifier is not needed, but may be added.

The double emulsions of the present invention may be used in food products, clinical products, pharmaceutical products, nutricosmetics, cosmetics, agro-chemical or other industrial products. Depending on the application, they may be freeze-dried or spray-dried. The dried form may be rehydrated in solution without losing the double emulsion texture.

Preferably, they are used in food products selected from salad dressing, mayonnaise-type products, sauces, spreads, soups, desserts, creams etc. The consistency and texture of these products may be tailored such that fluid, viscous or even thick textures are possible. In a preferred embodiment, the product may be a low-fat food product having the same sensory properties as the respective full fat version of the same product.

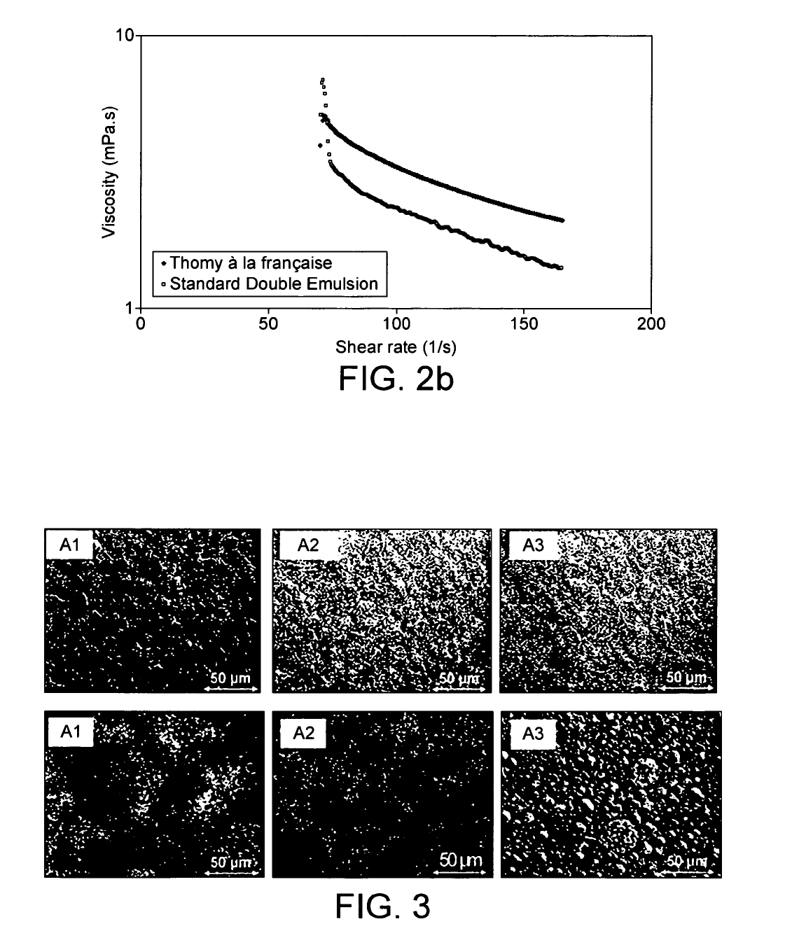

Referring to FIG. 2, it can be seen that the texture of the double emulsion of the present invention is close to the texture of a standard product “Thomy mayonnaise a la francaise”, especially in terms of yield stress before stirring (FIG. 2a) and viscosity at high shear rate (FIG. 2b). Indeed, the present double emulsion has a yield stress of 318 Pa at 0.9 of strain, versus a yield stress of 294 Pa at 0.6 of strain for the standard mayonnaise product. Similarly, the viscosity at 160 s⁻¹ shear rate is 1.425 for the present emulsion versus 2.16 for the standard product.

A tasting session has also revealed that the present double emulsions have been perceived as neutral, with a good texture similar to the full-fat mayonnaise. The double emulsion gave an impression of palate full-fat coating such as a mayonnaise.

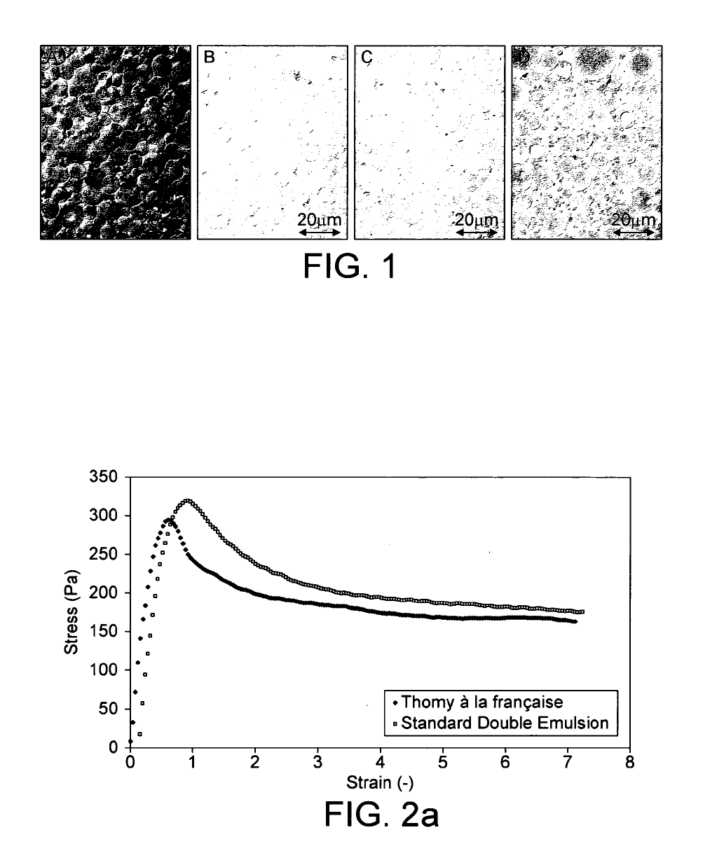

The double emulsion of the invention is very stable over time, with no phase separation or texture change over at least 1 month. Indeed, referring to FIG. 1, these images of the double emulsion of the invention are taken after sample preparation (A), 1 hour later (B), 1 week later (C), 1 month later (D). It can be seen that there is no structural change of the double emulsion over 1 month. The double emulsion consists of spherical droplets of oil in water emulsion of 1 to 10 microns in diameter. These droplets are almost close packed into the aqueous solution.

The present double emulsion may further comprise additional water-soluble molecules in the inner or in the outer aqueous phase of the double emulsion. The addition of these compounds does not compromise the texture of the final products enabling the production of a large variety of products, in particular low-fat products.

The double emulsions according to the invention are obtainable by a method described in the following.

The present method for the preparation of a double emulsion comprises, in a first step, the preparation of an internal aqueous phase comprising solutes. The solutes may be selected from salts, polyols and/or sugars. Typical salts include organic salts, sodium chloride, potassium chloride, calcium chloride, magnesium chloride, zinc chloride etc. Polyols or sugars which can be used in the present invention comprise any mono-, di- or oligosaccharides, starch, degradation products of starch, maltodextrins, dextran, glucose, sucrose, fructose, glycerol, polyglycerol, ethyleneglycol, propylene glycol etc.

The solutes concentration in the internal aqueous phase is preferably between 0.1 up to 30% by weight, more preferably 1% to 20% by weight, even more preferably 3% to 15% by weight.

To the internal aqueous phase is then added an oil phase comprising at least one emulsifier to obtain a well stabilised water-in-oil emulsion.

In the case of the use of a mixture of emulsifiers, it is such that each emulsifier has a differing molecular weight such that at least one emulsifier has a molecular weight of less than 2000 g/mol and at least a second emulsifier has a molecular weight of more than 700 g/mol. Preferably, at least one emulsifier has a molecular weight of less than 1800 g/mol, more preferably less than 1500 g/mol, even more preferably less than 1200 g/mol, while at least a second emulsifier preferably has a molecular weight of more than 800 g/mol, more preferably more than 1000 g/mol, even more preferably more than 1200 g/mol.

More preferably, the emulsifiers are such that at least one of the emulsifier is selected from the group of “low molecular weight” emulsifiers consisting of fatty acids, sorbitan esters, propylene glycol mono- or diesters, pegylated fatty acids, monoglycerides, derivatives of monoglycerides, diglycerides, pegylated vegetable oils, polyoxyethylene sorbitan esters, phospholipids, lecithin, cephalin, lipids, galactolipids, sugar esters, sugar ethers, sucrose esters, sorbitol anhydride monostearate, sorbitol anhydride monooleate, glycerol monooleate, or mixtures thereof and at least a second emulsifier is selected from the group of high molecular weight emulsifiers consisting of polyglycerol esters, polyglycerol polyricinoleic acid (PGPR), cellulose and its derivatives such as ethylcellulose, oil soluble proteins or peptides or hydrolysates, protein-polysaccharide complexes, coacervates or conjugates, food particles, fat particles, solid-lipid nanoparticles, micronised nutrient crystals, dietary fibres or mixtures thereof.

Most preferably, the emulsifier used to stabilise the inner water-in-oil emulsion is PGPR, glycerol monooleate (GMO) or a mixture thereof.

In a preferred embodiment, the amount of emulsifiers used to stabilise to water-in-oil emulsion is less than 5%, preferably less than 3%, more preferably less than 1% of the water-in-oil emulsion obtained.

The ratio of “low-molecular weight” emulsifier to “high-molecular weight” emulsifier is between 1:10 to 10:1 by weight. Preferably, it is between 1:5 and 5:1. Most preferably it is 4:1.

The weight percentage of the aqueous, solute containing phase in the water-in-oil emulsion may be from 1% to 80%, preferably, from 5% to 60%, and most preferable from 10% to 50%.

The conditions for forming the water-in-oil emulsions are standard conditions known to the skilled person. For instance, the mixture of oil and water is homogenised using high pressure or shear or a membrane or ultrasound, by mixers known in the art. The homogenisation speed and the duration of the shearing or mixing have an influence on the size of the water droplets inside the oil droplets, and thereby, on the texture and consistency of the final double emulsion.

Preferably, the conditions are such that the water droplets have a size between 70 nm and 20 microns. The size of the water droplets influences the consistency of the final double emulsion and may be adjusted accordingly. In a most preferred embodiment, the size of the water droplets is between 100 nm and 10 microns.

The last step in the present method is the mixing of the water-in-oil emulsion with an external aqueous phase to form a double emulsion. Preferably, the external aqueous phase comprises a hydrophilic polymer or polymer mixture or aggregate. In this way, the oil phase is stabilised in the external aqueous phase by said hydrophilic polymer.

Examples of such hydrophilic polymers or aggregates of polymers are amidated low methoxy pectin, caseinate, whey proteins, milk proteins, egg proteins, soy proteins, acacia gum, starch derivatives or other o/w emulsion stabilizing proteins or polysaccharides, or hydrophilic particles made out of protein aggregates, polysaccharide aggregates, hydrophilic particles made out of protein-surfactant aggregates, hydrophilic particles made out of protein-polysaccharide mixtures, hydrophilic particles made out of polysaccharide-polysaccharide mixtures, hydrophilic particles made out of polysaccharide-protein phase separating mixtures, or any mixtures thereof.

By hydrophilic particles is meant particles which are dispersible into an aqueous phase and show an interfacial activity. The term ‘interfacially active particles’ is described above.

Preferably, protein-polysaccharide mixtures, coacervates, conjugates, hybrids or particles are used as hydrophilic polymer in the present invention.

Most preferably, the hydrophilic polymer is taken in the group consisting of whey protein isolate, amidated low methoxy pectin, egg yolk or a mixture thereof.

The amount of external aqueous phase added to the water-in-oil emulsion is in the range of 15:85 up to 95:5. Preferably, it is in the range of 20:80 up to 80:20. More preferably, it is in the range of 30:70 up to 70:30.

In the present method, water-soluble molecules may further be included in the inner or in the outer aqueous phase of the double emulsion. The addition of these compounds does not compromise the texture of the final products enabling the production of a large variety of products, in particular low-fat products.

In a preferred embodiment of the present invention, the double emulsion obtained is allowed to set such as to form a textured double emulsion. This is due to the fact that the concentration of solutes in the internal aqueous phase is such that an osmotic pressure gradient is created between the internal and the external aqueous phases. Typically, several hours are needed for osmotic equilibration to occur between the phases. This final texture is found to be very stable over time and does no longer change. Thus, the double emulsion of the invention may be produced in a liquid form and poured or filled into a packaging without problem before the final viscosity is obtained. This provides a great advantage regarding ease of processing.

According to the present invention, the final consistency is controlled by a number of factors such as the amount and type of solutes present in the inner and outer aqueous phase, the amount of oil phase, the amount of inner water phase in the oil droplets, the size of the inner water phase droplets, the oil droplets size, the amount and type of emulsifier mixture used to stabilise the inner aqueous phase and the type of polymer used to stabilise the outer oil/water interface.

Most importantly, it has been found by the present invention that the type of emulsifiers used to stabilise the water-in-oil emulsion is crucial when stability of the whole double emulsion is concerned.

Thus, the use of least one emulsifier for stabilising a water-in-oil emulsion, wherein the water-in-oil emulsion is part of a double emulsion comprising said water-in-oil emulsion dispersed in an external aqueous phase also forms part of the present invention.

The emulsifiers are such as those described above with respect to the double emulsions and their method of production.

Most preferably, the emulsifiers are a mix of glycerol monooleate and polyglycerol polyricinoleic acid (PGPR).

The present invention thus provides a way in which double emulsions may be produced which are stable over time and which display consistencies which may be tailored to the desired use. In particular, low fat double emulsion may be obtained having the organoleptic properties of full fat products. In this way, a 10% fat mayonnaise-type product can be made using a double emulsion of the present invention, which is perceived as an 80% fat standard mayonnaise.

The present invention is further illustrated by means of the following non-limiting examples.

### Examples

The following procedure was used to form a number of products having mayonnaise-like textures, spread-like consistencies (e.g. Nutella type) or salad-dressing like consistencies.

A polymer is mixed into water with a rotor-stator mixer (Kinematic A G, Switzerland) at room temperature for 5 minutes to form an external aqueous phase. The solution is kept under agitation.

A homogeneous solution of sugar and/or salt in water is prepared at room temperature. A mixture of oil and emulsifiers is added to the solution at room temperature and the mixture is submitted to high shear (speed 3 to 10) with the rotor-stator mixer for 1 to 8 minutes to form a water-in-oil emulsion. The external aqueous phase is added to the water-in-oil emulsion or vice-versa, i.e. the water-in-oil emulsion is added to the external aqueous phase, and they are mixed together at room temperature under low shear (speed 3) for 1 minute. The sample is then stored at 4° C. for at least 1 to 6 hours prior to utilisation. During this time, the final consistency of the double emulsion evolves.

The samples may be sterilised and/or pasteurised using standard methods.

### Mayonnaise-Like Textures

| | Water-in-oil emulsion | Outer aqueous phase |
|---|---|---|
| Example 1 | | |
| Salt | 25% aqueous 1M NaCl | — |
| Sugar | 5% glucose | — |
| Emulsifiers | 8% PGPR | — |
| | 2% GMO | |
| Oil | 60% sunflower | — |
| Polymer | — | 5% WPI |
| | | 2% ALMP |
| Amount | 20% | 80% |
| Example 2 | | |
| Salt | 29% aqueous 1M NaCl | — |
| Sugar | 5% glucose | — |
| Emulsifiers | 4% Ethocel | — |
| | 2% GMO | |
| Oil | 60% sunflower | — |
| Polymer | — | 5% WPI |
| | | 2% ALMP |
| Amount | 20% | 80% |
| Example 3 | | |
| Salt | 24% aqueous 1M NaCl | — |
| Sugar | 4% glucose | — |
| Emulsifiers | 8% TiO₂ polymeric | — |
| | particles | |
| | 4% GMO | |
| Oil | 60% sunflower | — |
| Polymer | — | 5% WPI |
| | | 2% ALMP |
| Amount | 20% | 80% |
| Example 4 | | |
| Salt | 16% aqueous 2M NaCl | — |
| Sugar | 5% glucose | — |
| Emulsifiers | 2% PGPR | — |
| | 2% GMO | |
| Oil | 75% sunflower | — |
| Polymer | — | 6.25% WPI |
| | | 2.5% ALMP |
| Amount | 20% | 80% |
| Example 5 | | |
| Salt | 25% aqueous 1M NaCl | — |
| Sugar | 5% glucose | — |
| Emulsifiers | 8% PGPR | — |
| | 2% GMO | |
| Oil | 60% sunflower | — |
| Polymer | — | 6.25% WPI |
| Amount | 30% | 70% |
| Example 6 | | |
| Salt | 25% aqueous 1M NaCl | — |
| Sugar | 5% glucose | — |
| Emulsifiers | 8% PGPR | — |
| | 2% GMO | |
| Oil | 60% sunflower | — |
| Polymer | — | 6.25% WPI |
| Amount | 40% | 60% |
| Example 7 | | |
| Salt | 5% aqueous 1M NaCl | — |
| Sugar | 1% glucose | — |
| Emulsifiers | 1.6% PGPR | |
| | 0.4% GMO | |
| Oil | 92% sunflower | — |
| Polymer | — | 6.25% WPI |
| | | 2.5% ALMP |
| Amount | 40% | 60% |
| Example 8 | | |
| Salt | 10% aqueous 1M NaCl | — |
| Sugar | 2% glucose | — |
| Emulsifiers | 3.2% PGPR | — |
| | 0.8% GMO | |
| Oil | 60% sunflower | — |
| Polymer | — | 6.25% WPI |
| | | 2.5% ALMP |
| Amount | 30% | 70% |
| Example 9 | | |
| Salt | 8% aqueous 4M NaCl | — |
| Sugar | 5% glucose | — |
| Other | 6.75% vinegar (10%) | 0.37% vinegar (10%) |
| | 1.25% water | 2.5% mustard |
| | | 5% egg yolk water |
| | | solution |
| Emulsifiers | 2% PGPR | — |
| | 2% GMO | |
| Oil | 75% sunflower | — |
| Polymer | — | 6.25% WPI |
| | | 2.5% ALMP |
| Amount | 30% | 70% |
| Example 10 | | |
| Salt | 3% NaCl | — |
| Sugar | — | — |
| Other | 1.5% vinegar (10%) | 10% vinegar (10%) |
| | 25.5% water | 10% mustard |
| | | 39% FGEM egg yolk |
| Emulsifiers | 0.6% PGPR | — |
| Oil | 69.4% sunflower | — |
| Polymer | — | — |
| Amount | 65% | 35% |
| Example 11 | | |
| Salt | 3% NaCl | — |
| Sugar | — | — |
| Other | 1.5% vinegar (10%) | 10% vinegar (10%) |
| | 25.5% water | 10% mustard |
| | | 39% FGEM egg yolk |
| Emulsifiers | 0.5% PGPR | — |
| Oil | 69.5% sunflower | — |
| Polymer | — | 1% ALMP |
| Amount | 70% | 30% |

### Spread-Like Consistencies

| Example 12 | Water-in-oil emulsion | Outer aqueous phase |
|---|---|---|
| Salt | 10% aqueous 1M NaCl | — |
| Sugar | 2% glucose | — |
| Emulsifiers | 3.2% PGPR | — |
| | 0.8% GMO | |
| Oil | 84% sunflower | — |
| Food-grade polymer | — | 6.25% WPI |
| | | 2.5% ALMP |
| Amount | 40% | 60% |

Salad-Dressing Like Consistencies

| | Water-in-oil emulsion | Outer aqueous phase |
|---|---|---|
| Example 13 | | |
| Salt | 5% aqueous 1M NaCl | — |
| Sugar | 1% glucose | — |
| Emulsifiers | 1.6% PGPR | — |
| | 0.4% GMO | |
| Oil | 92% sunflower | — |
| Polymer | — | 6.25% WPI |
| | | 2.5% ALMP |
| Amount | 20% | 80% |
| Example 14 | | |
| Salt | 5% aqueous 1M NaCl | — |
| Sugar | 1% glucose | — |
| Emulsifiers | 1.6% PGPR | — |
| | 0.4% GMO | |
| Oil | 92% sunflower | — |
| Polymer | — | 6.25% WPI |
| | | 2.5% ALMP |
| Amount | 30% | 70% |
| Example 15 | | |
| Salt | 10% aqueous 1M NaCl | — |
| Sugar | 2% glucose | — |
| Emulsifiers | 3.2% PGPR | — |
| | 0.8% GMO | |
| Oil | 84% sunflower | — |
| Polymer | — | 6.25% WPI |
| | | 2.5% ALMP |
| Amount | 20% | 80% |
| Example 16 | | |
| Salt | 10% aqueous 1M NaCl | — |
| Sugar | 2% glucose | — |
| Emulsifiers | 2% PGPR | — |
| | 0.5% GMO | |
| Oil | 85.5% sunflower | — |
| Food-grade polymer | — | 6.25% WPI |
| Amount | 20% | 80% |
| Example 17 | | |
| Salt | 25% aqueous 1M NaCl | — |
| Sugar | 5% glucose | — |
| Emulsifiers | 2% PGPR | — |
| | 0.5% GMO | |
| Oil | 67.5% sunflower | — |
| Food-grade polymer | — | 6.25% WPI |
| Amount | 30% | 70% |

PGPR: polyglycerol polyricinoleic acid (PGPR 90)
Ethocel 45: Ethylcellulose from Dow Chemicals
GMO: glycerol monooleate (Dimodan MO90, Danisco)
TiO₂: Titanium dioxide particles
WPI: whey protein isolate (Bipro, U.S.)
ALMP: amidated low methoxy pectin (Grinsted Pectin LA040)
FGEM egg yolk: factory grade enzyme modified egg yolk.

1. Double emulsion comprising an internal aqueous phase dispersed in an oil phase forming a water-in-oil emulsion, the water-in-oil emulsion being dispersed in an external aqueous phase,
the water-in-oil emulsion comprises at least one emulsifier, the internal aqueous phase comprises solutes and the external aqueous phase comprises at least one hydrophilic polymer or polymer aggregates.

2. Double emulsion according to claim 1, the water-in-oil emulsion comprises at least two different emulsifiers having different molecular weights.

3. Double emulsion according to claim 1, comprising one emulsifier having a molecular weight of less than 2000 g/mol and having a second emulsifier having a molecular weight of more than 700 g/mol.

4. Double emulsion according to claim 1, wherein at least one emulsifier has a molecular weight of less than 1800 g/mol, and at least a second emulsifier has a molecular weight of more than 800 g/mol.

5. Double emulsion according to claim 1, wherein at least one emulsifier is selected from the group consisting of low molecular weight emulsifiers consisting of fatty acids, sorbitan esters, propylene glycol mono- or diesters, pegylated fatty acids, monoglycerides, derivatives of monoglycerides, diglycerides, pegylated vegetable oils, polyoxyethylene sorbitan esters, phospholipids, lecithin, cephalin, lipids, sugar esters, sugar ethers, sucrose esters, sorbitol anhydride monostearate, sorbitol anhydride, monooleate, glycerol monooleate, and mixtures thereof and at least a second emulsifier is selected from the group consisting of high molecular weight emulsifiers consisting of polyglycerol esters, polyglycerol polyricinoleic acid (PGPR), cellulose and its derivatives, oil soluble proteins, peptides, hydrolysates, protein-polysaccharide complexes, coacervates, conjugates, food particles, fat particles, solid-lipid nanoparticles, micronised nutrient crystals, dietary fibres and mixtures thereof.

6. Double emulsion according to claim 1, wherein the polymer or aggregate is selected from the group consisting of amidated low methoxy pectin, caseinate, whey proteins, milk proteins, egg proteins, egg yolk, soy proteins, acacia gum, starch derivatives, emulsion stabilizing proteins, polysaccharides, hydrophilic particles made out of protein aggregates, polysaccharide aggregates, hydrophilic particles made out of protein-surfactant aggregates, hydrophilic particles made out of protein-polysaccharide mixtures, hydrophilic particles made out of polysaccharide-polysaccharide mixtures, hydrophilic particles made out of polysaccharide-protein phase separating mixtures, and any mixtures thereof.

7. Double emulsion according to claim 6, wherein the polymer is selected from the group consisting of whey protein isolate, amidated low methoxy pectin, egg yolk and a mixture thereof.

8. Double emulsion according to claim 1, wherein the solutes are selected from the group consisting of salts, polyols and sugars.

9. Double emulsion according to claim 1, wherein the internal aqueous phase is under osmotic pressure.

10. Double emulsion according to claim 1, wherein the external aqueous phase is not gelified.

11. Double emulsion according to claim 1, wherein the double emulsion comprises 1% to 55%.

12. Double emulsion according to claim 1, wherein the double emulsion comprises a viscosifier selected from the group consisting of amidated low methoxy pectin, pectin, alginates, carrageenan, locust bean gum, guar gum, tragacanth, Acacia gum, xanthan gum, Karaya gum, gellan gum, polydextrose, dextrin, modified starch, oxidized starch, cellulose derivatives, and mixtures thereof.

13. Method for the preparation of a double emulsion comprising the steps of:
preparing an internal aqueous phase comprising solutes
combining the internal aqueous phase with an oil phase comprising at least one emulsifier to obtain a stabilised water-in-oil emulsion; and
combining the water-in-oil emulsion with an external aqueous phase to form a double emulsion, the external aqueous phase comprises at least one hydrophilic polymer or polymer aggregates.

14. Method according to claim 13, wherein the solutes concentration in the internal aqueous phase is such that an osmotic pressure gradient is created between the internal and the external aqueous phases.

15. Method according to claim 13, wherein the double emulsion obtained in step c is allowed to set such as to form a textured double emulsion.

16. Method according to claim 13, wherein the at least one emulsifier has a molecular weight of less than 2000 g/mol and at least a second emulsifier has a molecular weight of more than 700 g/mol.

17. Method according to claim 13, wherein at least one emulsifier has a molecular weight of less than 1800 g/mol, and at least a second emulsifier has a molecular weight of more than 800 g/mol.

18. Method according to claim 13, wherein at least one emulsifier is selected from the group consisting of low molecular weight emulsifiers consisting of fatty acids, sorbitan esters, propylene glycol mono- and diesters, pegylated fatty acids, monoglycerides, derivatives of monoglycerides, diglycerides, pegylated vegetable oils, polyoxyethylene sorbitan esters, phospholipids, lecithin, cephalin, lipids, galactolipids, sugar esters, sugar ethers, sucrose esters, sorbitol anhydride monostearate, sorbitol anhydride monooleate, glycerol monooleate, and mixtures thereof and at least a second emulsifier is selected from the group consisting of high molecular weight emulsifiers consisting of polyglycerol esters, polyglycerol polyricinoleic acid (PGPR), cellulose and its derivatives, oil soluble protein, peptides, hydrolysates, protein-polysaccharide complexes, coacervates, conjugates, food particles, fat particles, solid-lipid nanoparticles, micronised nutrient crystals, dietary fibres and mixtures thereof.

19. Method according to claim 13, wherein the solutes are selected from the group consisting of salts, polyols and sugars.

20. Method according to claim 13, wherein the external aqueous phase comprises a viscosifier selected from the group consisting of amidated low methoxy pectin, pectin, alginate, carrageenan, locust bean gum, guar gum, tragacanth, Acacia gum, xanthan gum, Karaya gum, gellan gum, polydextrose, dextrin, modified starch, oxidized starch, and mixtures thereof.

21. Method according to claim 13, wherein the double emulsion is spray-dried or freeze-dried.

22. Double emulsion obtainable by the method of claims 13.

23. Use of a mixture of at least two different emulsifiers having differing molecular weights for stabilising a water-in-oil emulsion that is part of a double emulsion comprising the water-in-oil emulsion dispersed in an external aqueous phase.

24. Use according to claim 24, wherein one emulsifier has a molecular weight of less than 2000 g/mol and a second emulsifier has a molecular weight of more than 700 g/mol.

25. Use according to claim 24, wherein one emulsifier has a molecular weight of less than 1800 g/mol and a second emulsifier has a molecular weight of more than 800 g/mol.

26. Use according to claim 24, wherein at least one emulsifier is selected from the group consisting of low molecular weight emulsifiers consisting of fatty acids, sorbitan esters, propylene glycol mono- or diesters, pegylated fatty acids, monoglycerides, derivatives of monoglycerides, diglycerides, pegylated vegetable oils, polyoxyethylene sorbitan esters, phospholipids, lecithin, cephalin, lipids, galactolipids, sugar esters, sugar ethers, sucrose esters, sorbitol anhydride monostearate, sorbitol anhydride monooleate, glycerol monooleate, and mixtures thereof and at least a second emulsifier is selected from the group consisting of high molecular weight emulsifiers consisting of polyglycerol esters, polyglycerol polyricinoleic acid (PGPR), cellulose and its derivatives, oil soluble proteins, peptides, hydrolysates, protein-polysaccharide complexes, coacervates, conjugates, food particles, fat particles, solid-lipid nanoparticles, micronised nutrient crystals, dietary fibres and mixtures thereof.

27. Use according to claim 24, wherein the emulsifiers are glycerol monooleate and polyglycerol polyricinoleic acid (PGPR).

28. Use of a double emulsion comprising an internal aqueous phase dispersed in an oil phase forming a water-in-oil emulsion, the water-in-oil emulsion being dispersed in an external aqueous phase,
the water-in-oil emulsion comprises at least one emulsifier, the internal aqueous phase comprises solutes and the external aqueous phase comprises at least one hydrophilic polymer or polymer aggregates in a product selected from the group consisting of food products, clinical products, pharmaceutical products, nutricosmetics, cosmetics, agro-chemical and other industrial products.

29. Use according to claim 29, wherein the food product is selected from the group consisting of salad dressing, mayonnaise-type products, sauces, spreads, soups, desserts, and creams.
